(19) United States
(12) Patent Application Publication
(10) Pub. No.: US 2007/0102503 A1
(43) Pub. Date: May 10, 2007

(54) Virtual device for counting, timing, recording, and charting fetal movement frequency

(76) Inventor: Diep Mong Nguyen, Manhattan Beach, CA (US)

Correspondence Address: BUCHANAN, INGERSOLL & ROONEY PC, POST OFFICE BOX 1404, ALEXANDRIA, VA, 22313-1404, US

(21) Appl. No.: 11/400,563

(22) Filed: Apr. 7, 2006

Related U.S. Application Data
(63) Continuation-in-part of application No. 11/219,405, filed on Sep. 2, 2005.

(51) Int. Cl. G01G 23/18; G06M 1/06; A61B 8/00; A61B 5/103

(52) U.S. Cl. 235/1 R; 600/595; 600/453

(57) ABSTRACT

Methods, systems, and computer readable media for kick counting and charting fetal movements. The virtual kick counting device records ten fetal movements in a session, wherein each movement is recorded by pressing a button included in a device image displayed on a computer display monitor. The virtual device displays an additional footprint icon each time the button is pressed, wherein the user can count the recorded movements during the session. The virtual device records the elapsed time (KickTime) for each session and stores the KickTime. The virtual device has a sound recording and playback function that enables the user to record the fetal heartbeat sound for playback. The virtual device stores and updates the weeks and days of the mother's pregnancy progress, calculates the countdown days-to-go, and displays the countdown if the countdown is less than 100.

### CROSS REFERENCE TO RELATED APPLICATIONS

This application is a continuation-in-part of U.S. application Ser. No. 11/219,405, entitled “A device and methods for counting, timing, recording, and charting fetal movement frequency,” and filed on Sep. 02, 2005.

### FIELD OF THE INVENTION

The present invention relates to computer systems, more particularly but not exclusively to techniques for helping a pregnant woman count and chart fetal movements.

### BACKGROUND OF THE INVENTION

Perceived fetal movement by a pregnant woman is regarded as an excellent indication of fetal well-being. Fetal movement serves as an indirect measure of the central nervous system integrity and function. Although fetal movement can be documented by ultrasound as early as 7-8 weeks of pregnancy, the first fetal movement or “flutter” is not usually felt by the mother until the 16th week (for women who have delivered a baby) to the 20th week (for women pregnant for the first time). Movements generally increase in strength and frequency through pregnancy, particularly at night, and when the woman is at rest. At the end of pregnancy (36 weeks and beyond), there is normally a slow change in movements, with fewer violent kicks and more rolling and stretching fetal movements.

Maternal counting and tracking of fetal movements is called kick count. It has been accepted as a cheap, simple, valuable, effective, reliable and harmless screening of fetal well-being in both low and high risk pregnancies. For example, a sudden decrease in fetal movements may indicate potential problems and may require further evaluation of fetal well-being. A study being carried out by the International MOMSTUDY (http://www.MOMStudy.com), currently with more than 3000 participants, has found that more than half of stillbirth mothers (nearly three quarter had unexplained stillbirths in the third trimester) reported their first reason to believe that their baby was not doing well was a reduction in fetal activity. Half of the affected mothers had perceived a gradual reduction of fetal movement several days before.

Several formal protocols for kick count have been developed over the years. However, there is no single accepted protocol. For example, Pearson and Weaver's study (“British Medical Journal,” Vol. 1, pp. 1305-1307, May 29, 1976) suggested counting 10 fetal movements daily and reporting if there are less than 10 movements in 12 hours. Sadovsky et al. (“Obstetrics and Gynecology,” Vol. 50, No. 1, pp. 49-55, July 1977) suggested counting fetal movements for 30 minutes to one hour, three times a day, and if there are less than 3 movements in one hour, the count is continued for 6-12 hours and reporting if there are less than 10 movements in 12 hours, while Neldam's study (“The Lancet,” pp. 1222-1224, Jun. 7, 1980,) suggested counting fetal movement for 2 hours after a meal, 3 times a week and reporting if there are less than 3 movements per hour. In Moore and Piacquadio's study (“American Journal of Obstetrics and Gynecology,” Vol. 160, pp. 1075-1080, May 1989), the fetal mortality rate significantly fell from 8.7 to 2.1 per 1,000 deliveries by counting fetal movements and taking prompt actions for further evaluation of fetal well-being when the pregnant women did not perceive 10 movements within a two hour time frame. The mean interval time for 10 perceived fetal movements was 20.9+/−18.1 minutes (mean+/−standard deviation). The number of antepartum testing (nonstress test, contraction stress test, biophysical profile) prompted by fetal movement count increased by 13%. Regardless of the counting methods used, this and other existing studies suggested an improvement in perinatal outcomes with early identification of decreased fetal activity. There are other on-going research activities which support the benefit of kick count, the details of which are readily available to those of ordinary skill and are not discussed here for simplicity.

In general, the kick count may be carried out at approximately the same time each day, preferably when the baby is usually very active or after mother has had a meal or snack. The mother may note the starting time and begin counting fetal movements (rolls, kicks, punches, turns), excluding hiccups, and continue counting until a certain number of movements have been noted. Following the Moore and Piacquadio's study, perception of 10 distinct movements in a period of up to 2 hours may be considered reassuring. Once ten movements have been perceived, the count may be discontinued.

Regardless of the type of protocol selected for a kick count, the efficacy of the kick count is affected by the accuracy in recording fetal movements during the kick count. Traditionally, a kick count chart was used where the information about the kick count session was recorded manually and, as a consequence, noncompliance has been a significant issue. As such, there is a need for a simple, user friendly device for accurate kick counting and for helping the user to record the time taken for a predetermined number of fetal movements. The user may report the recorded kick times to the prenatal care clinic or obstetrical care to check the fetal health conditions.

### OBJECTIVES OF THE INVENTION

It is an object of this invention to provide methods of kick count, in particular counting, timing and recording a predetermined number of fetal movements using the count-to-ten protocol, e.g., 10 fetal movements over 2 hours, as a means for fetal well-being monitoring.

It is another object of this invention to provide a simple method of fetal movement counting and charting that can be used at any time of the day, during the third trimester of pregnancy to enhance the compliance with the kick count protocol.

It is yet another object of this invention to incorporate voice and audio technology to simplify fetal movement counting.

It is still another object of this invention to provide an incentive for a pregnant woman to perform fetal movement counting by incorporating automatic audio playback of the fetal heart sounds at the end of each counting session. The invention also incorporates a counter into which the user can enter the pregnancy progress, in weeks and days and update it at any time.

It is another object of this invention to provide a means for the user to record audio, which can be played back at the end of the kick session. This audio file can be retrieved and e-mailed to friends and family.

It is another object of this invention to provide a means for importing audio files recorded with other devices, for example the obstetrical provider's Doppler equipment.

It is another object of this invention to provide a means for e-mailing the kick count sessions to the user's obstetrical provider.

### SUMMARY OF THE INVENTION

The present invention provides methods for charting fetal movements by use of a virtual kick counting device (or, shortly, virtual device) that is generated by computer software and displayed on a computer display monitor. Typically, the term “virtual device” refers to computer software for simulating an actual device by use of a computer. Hereinafter, the term “virtual device” is used to collectively refer to a system including a device image displayed on the monitor as a graphic user interface, computer software and pertinent components of the computer for simulating the functions of an actual kick counting device. The user of the virtual device records each movement (or kick) by pressing a virtual button or button image included in the device image. The virtual device records the time interval between the first kick and the tenth kick in each kick count session, where the elapsed time interval is referred to as “KickTime” hereinafter. The virtual device stores ten KickTimes so that the user can review the frequency of the kicks from one session to the next. In addition, the virtual device has a sound recording and playback function that allows the user to record the fetal heartbeat sound for playback. The virtual device includes audible instructions to inform the user how to operate the device in each mode. These instructions may also be provided as text that visually instructs the user. The virtual device also stores and updates the weeks and days of the woman's pregnancy progress.

In one aspect of the present invention, a method to be performed by a computer for charting fetal movements includes steps of: displaying a device image on the screen of the computer, the device image including an image of an input member (or, equivalently, a button image) actuatable by a user to input fetal movement information into the computer, the information corresponding to one or more perceived fetal movements; recording the fetal movement information during a kick count session, the kick count session being a period for recording a preset number of fetal movements; and displaying icons on the device image during the kick count session, each icon corresponding to one of the perceived fetal movements. The user is able to count the perceived fetal movements recorded during the kick count session by counting the icons displayed on the device image during the kick count session.

These and other features, aspects and advantages of the present invention will become better understood with reference to the following drawings, description and claims.

### BRIEF DESCRIPTION OF THE DRAWINGS

FIG. 1 shows a schematic diagram of an exemplary computer that may be used in embodiments of the present invention.

FIG. 2 shows an exemplary device image displayed on a computer display monitor in accordance with a preferred embodiment of the present invention.

FIG. 3A is an exemplary device image in a “kick” mode for recording the times of fetal movements in accordance with the preferred embodiment of the present invention.

FIG. 3B is an exemplary device image in a “play animation” mode for playing an animation upon completion of the kick mode in accordance with the preferred embodiment of the present invention.

FIG. 3C is an exemplary device image in a “kick memory” mode for reviewing KickTimes recorded in the kick mode in accordance with the preferred embodiment of the present invention.

FIG. 3D is an exemplary device image in a “play” mode for playing a fetal heartbeat sound in accordance with the preferred embodiment of the present invention.

FIG. 3E is an exemplary device image in a “record” mode for recording a fetal heartbeat sound to be played in the play mode in accordance with the preferred embodiment of the present invention.

FIG. 3F is an exemplary device image in a “setup” mode for inputting information of the current stage of pregnancy, selecting a language, and updating a “Days To Go” countdown in accordance with the preferred embodiment of the present invention.

FIGS. 4A-4B show a flow chart illustrating an operational sequence of the modes depicted in connection with FIGS. 3A-3F.

FIGS. 5A-5C show a system flowchart of the virtual device depicted in connection with FIGS. 1-3F.

FIG. 6 shows a flow chart illustrating the steps that may be carried out to count kicks using the virtual device in accordance with the preferred embodiment of the present invention.

### DETAILED DESCRIPTION OF THE INVENTION

In the present disclosure, numerous specific details are provided, such as examples of apparatus, components, and methods, to provide a thorough understanding of embodiments of the invention. Persons of ordinary skill in the art will recognize, however, that the invention can be practiced without one or more of the specific details. In other instances, well-known details are not shown or described to avoid obscuring aspects of the invention.

Being computer-related, it can be appreciated that the components disclosed herein may be implemented in hardware, software, or a combination of hardware and software (e.g., firmware). Software components may be in the form of computer-readable program code stored in a computer-readable storage medium, such as memory, mass storage device, or removable storage device. For example, a computer-readable storage medium may comprise computer-readable code for performing the function of a particular component. Likewise, computer memory may be configured to include one or more components, which may then be executed by a processor. Components may be implemented separately in multiple modules or together in a single module.

Broadly, the present invention provides a virtual kick counting device (or, shortly, virtual device) that is intended to help a pregnant woman chart fetal movements. The virtual device may help the pregnant woman record and evaluate the elapsed time for ten perceived fetal movements to occur in a kick count session. The virtual device may include a device image displayed on a computer display monitor. The user of the virtual device may record each movement (or kick) by pressing a button image included in the device image. The time between the first recorded kick and the tenth is measured by the device and called the ‘Kick Time’. The virtual device may store data of ten KickTimes so that the user can review the frequency of the kicks from one sequence to the next. In addition, the virtual device may have a sound recording and playback function that enables the user to record and play a fetal heartbeat sound (or any other sound they decide to record). The virtual device may also store and update the weeks and days of the woman's pregnancy progress.

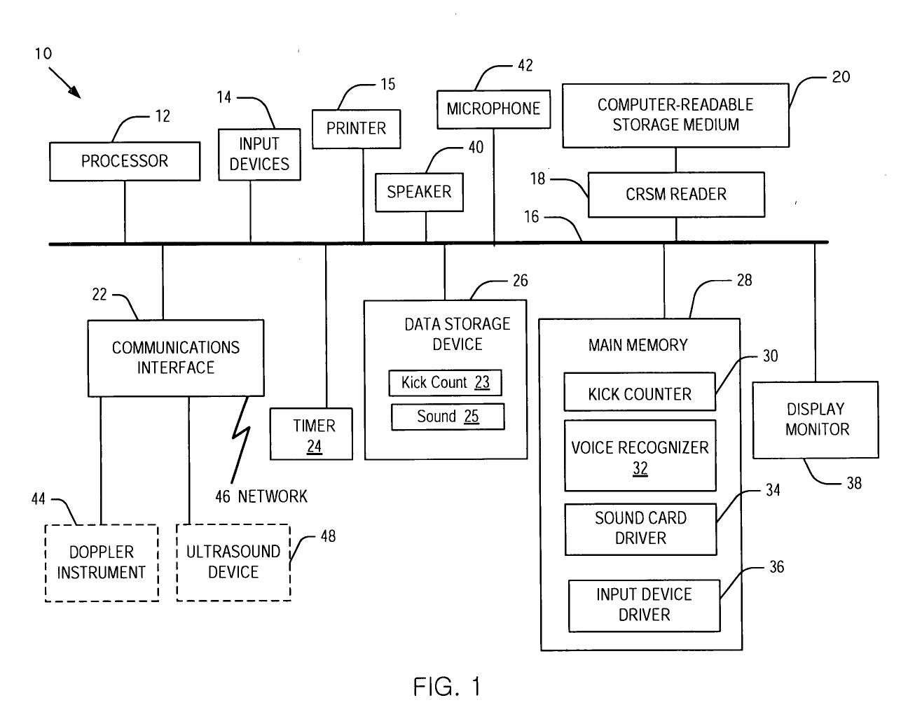

Referring now to FIG. 1, there is shown a schematic diagram of an exemplary computer shown at 10 that may be used in embodiments of the present invention. The computer shown at 10 may have less or more components to meet the needs of a particular application. As shown in FIG. 1, the computer shown at 10 may include processor 12, such as those from the Intel Corporation or Advanced Micro Devices, for example. The computer may also include one or more buses 16 coupling its various components, and one or more input devices 14 (e.g., keyboard, mouse) for enabling the user to interact with the computer. The computer 10 may further include a computer-readable storage medium (CRSM) 20, such as CD or USB storage device, a CRSM reader 18 (e.g., floppy drive, CD-ROM drive), a display monitor 38 (e.g., cathode ray tube, flat panel display) for displaying images, a communication interface 22 (e.g., network adapter, modem) for coupling to network 46, one or more data storage devices 26 (e.g., hard disk drive, optical drive, FLASH memory), main memory 28 (e.g., RAM), a speaker 40, and a timer 24 for measuring the lapse of time between two events and providing clock signals or clock times. The computer may have various peripheral devices, such as a printer 15. As will be describe later, the communication interface 22 may be also connected to optional instruments, such as a Doppler instrument 44 and an ultrasound device 48.

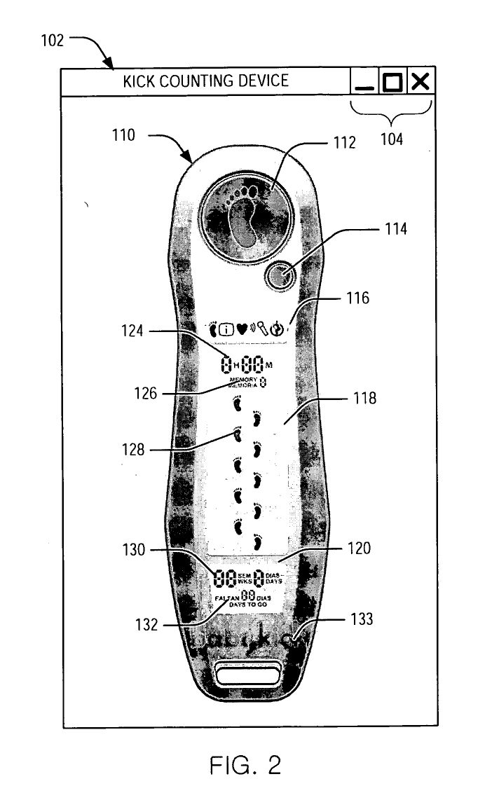

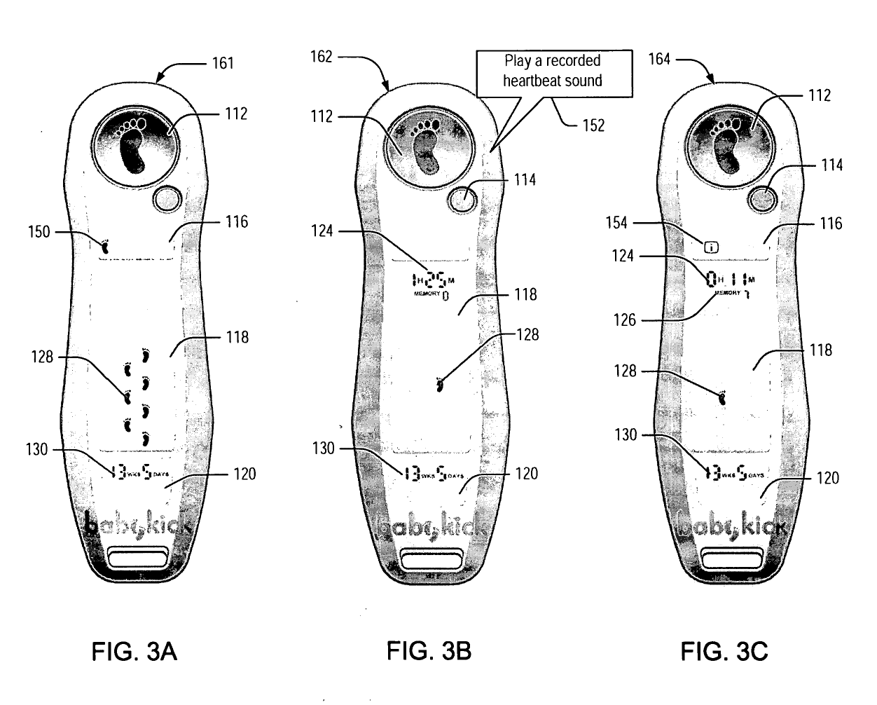

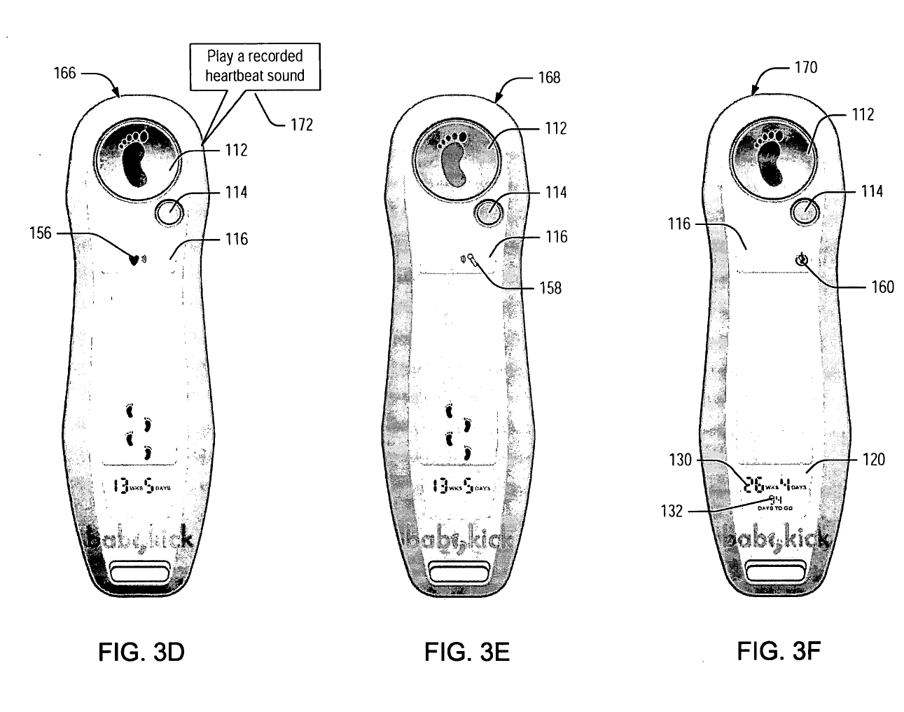

Software embodiments may be stored in a computer-readable storage medium 20 for reading into the data storage device 26 or main memory 28. In the example of FIG. 1, main memory 28 may be configured to include a kick counter 30, a voice recognizer 32, a sound card driver 34, and an input device driver 36. The kick counter 30 may be a software program and the heart of the virtual device. As such, the term “virtual device” may be used interchangeably with the term “kick counter.” The kick counter 30 may control the display of device images (FIGS. 2-3F) and orchestrate operations of the computer components to realize various functions of the virtual device. The kick counter 30 may be installed in the computer 10 via the network 46, such as the Internet, or by use of the CRSM 20 or any other suitable electronic devices, such as PDA and cell phone. The voice recognizer 32 may be a software program to recognize the user's command to operate the virtual device. The sound card driver 34 may be a software program operative to receive sound from an audio member, such as microphone 42, coupled to the computer 10 by use of a sound card (not shown in FIG. 1), operative to store the received sound into the data storage device 26, and operative to play the stored sound on the speaker 40 upon request of the user. The input device driver 36 may be a software program to operate the input devices 14. For instance, when the user clicks a mouse button, the input device driver 36 may act as an interruption/event handler and respond to send a signal to the kick counter 30 that subsequently transmits a proper command signal to a corresponding component of the computer 10. The data storage device 26 may be used to store various data including kick count information (such as times of fetal movements and KickTime) 23 and sound recording 25 received through the microphone 42 and/or the communication interface 22. The data storage device 26 may also store computer software including the kick counter 30, voice recognizer 32, sound card driver 34 and input device driver 36, wherein the computer software may be copied into the main memory 28 when the computer 10 is booted up. It should be apparent to those of ordinary skill that the communication interface 22 may be used to communicate various types of audio/video data, for instance, heartbeat sound and sonographic images, to the computer 10. The data exchanged through the communication interface 22 may be in the form of email or formatted in accordance with other suitable communication protocols. The computer 10 may be operated by a remote user, such as a system administrator located remotely from the computer 10, via the network 46 and the communication interface 22.

FIG. 2 shows a device image 110 displayed on the computer display monitor 38 in accordance with the present invention, wherein the device image 110 is similar in appearance to a previous embodiment of the actual kick counting device described in U.S. application Ser. No. 11/219,405, which is the parent application of the present application and incorporated by reference in its entirety. The device image 110 may be generated by the kick counter 30. This device image 110 may be customized by the user, for the user could change the color of the displayed kick counter image to suit their preference. This option could be offered by right clicking over the kick counter image and selecting ‘color’ from a drop down menu. As illustrated, the device image 110 may include: an input member including an EVENT button 112 for recording events and a MODE button 114 for switching between modes; a mode window 116 for indicating the active mode; a kick count window 118 that includes a time display 124, a memory number display 126, and a 10-footprint display 128; a pregnancy progress window 120 that includes a week-and-day counter 130; and a day counter 132. (Hereinafter, the terms footprints and feet are used interchangeably.). It is noted that each component of the device image 110 refers to an image of the counterpart component of the actual kick counting device. For instance, the EVENT button 112 in FIG. 2 refers to an image of the EVENT button of the actual kick counting device.

The week-and-day counter 130 may indicate a count forward of the pregnancy in weeks and days from the inception of pregnancy, while the day counter 132 may indicate a countdown of ‘days-to-go’ during the last 99 days of the pregnancy. As will be explained later, the words “DAYS TO GO” of the day counter 132 may also be used to select the language by the user of the virtual device. Also, a device name 133 may be shown on the device image 110.

The computer 10 may display an icon (not shown in FIG. 2) on the display monitor 38 when booted, indicating the virtual device is running in the main memory 28. The user may click the icon to open a window 102 that includes the device image 110. The user may minimize, maximize, or close the window 102 by clicking the boxes 104. It is noted that the configuration of the window 102 shown in FIG. 2 is illustrative such that other configuration may be contemplated for different computer operating systems. The user may be required to log into the virtual device by selecting her user name as the virtual instrument starts up. This would allow more than one user to use the virtual device on a given computer. Her kick count data would then be saved for future reference.

The user may operate (or, equivalently, press) the EVENT button 112 and MODE button 114 by a conventional method, i.e., the user may move the mouse cursor over one of the buttons and click a mouse button. Or, the user may press a preset key(s) of an input device 14, such as a keyboard, to operate the buttons 112 and 114 on the device image 110.

The virtual device (or, equivalently, the kick counter 30) may have the following (operational) modes; kick mode, play animation mode, kick memory mode, play mode, record mode and setup mode. The user may operate the device image 110 to get audible instructions for each mode that will play after a predetermined time delay. These instructions may be stored in the data storage device 26 and inform the user in the correct language how to operate the virtual device in that particular mode. These instructions may also be text prompts that appear on the screen. The user of the virtual device may press the MODE button 114 to switch from one mode to another while one of the icons in the mode window 116 may be highlighted to indicate the current mode. FIG. 3A is an exemplary device image 161 in a “kick” mode for recording the times of fetal movements. The virtual device may default to the kick mode when awaken from an idle state. As illustrated, a kick icon 150 may be highlighted in the mode window 116 to indicate that the virtual device is currently operating in the kick mode. In this mode, the user of the virtual device, preferably the mother, may record fetal movements (or, shortly, kicks) by pressing the EVENT button 112 each time that the mother perceives a fetal movement. When the EVENT button 112 is pressed, the input device driver 36 may send a signal to the kick counter 30 so that the kick counter 30 may store the time of fetal movement in the data storage device 26. In the kick mode (and other modes described in FIGS. 3B-3F), the week-and-day counter 130 of the pregnancy progress window 120 may display a count forward from inception in weeks and days. If the current date is in the last 99 days of the pregnancy, the day counter 132 may display a count down to delivery in days as shown in FIG. 2. The display on the week-and-day counter 130 and the day counter 132 may be controlled by the kick counter 30.

In each (kick count) session, the user may record ten consecutive kicks. To indicate the number of kicks recorded in the current session, the kick count window 118 may display the footprint image 128. When a kick is recorded in the data storage device 26 by pressing the EVENT button 112, another image of the footprint 128 may be added to the kick count window 118. After ten kicks have been recorded, the time taken to count these ten kicks (or equivalently, KickTime) may be displayed on the time display 124 of the kick window 118, as illustrated in FIG. 3B. FIG. 3B is an exemplary device image 162 in a “play animation” mode. Upon completion of the kick mode in FIG. 3A, the virtual device may default to the play animation mode, wherein the animated footprints 128 may walk up the kick window 118 along with a heartbeat sound for ten seconds as indicated in a text bubble 152. To be more specific, the kick counter 30 may send a first signal to sound card driver 34 to play the heartbeat sound on the speaker 40 and, at the same time, send a second signal to the display monitor 38 to display the animation of the footprints 128. The fetal heartbeat sound may be recorded in the “record” mode and stored in the data storage device 26 as will be explained in connection with FIG. 3E. The play animation mode may alert the user that ten kicks have been recorded and one session is over.

As mentioned above, the user may record ten kicks in each session. The number “ten” has been selected to utilize the protocol recommended by American College of Obstetricians and Gynecologists (ACOG): 10 fetal movements (kicks) in 2 hours. However, it should be apparent to those of ordinary skill that the virtual device may be designed to record other suitable number of fetal movements in each session depending upon the kick count protocol.

Upon completion of the kick mode (FIG. 3A), the virtual device may enter into the play animation mode (FIG. 3B) automatically. Likewise, once the animation is complete, the virtual device may default to a “kick memory” (or, shortly “memory”) mode. FIG. 3C is an exemplary device image 164 in the kick memory mode for reviewing the KickTimes recorded in the kick mode in FIG. 3A. As illustrated in FIG. 3C, a memory icon 154 may be highlighted in the mode window 116 to indicate that the virtual device is currently operating in the kick memory mode. In this mode, the device image 164 may display ten KickTime records sequentially, one at a time. The EVENT button 112 may be used to scroll through the ten KickTime records that may be stored in the data storage device 26. For each KickTime record displayed, the corresponding memory number (or equivalently, a stack number of the displayed KickTime record in the ten KickTime memory) may be displayed on the memory number display 126. More specifically, the kick counter 30 may access the data storage device 26 to retrieve the KickTime records and send appropriate signals to the display monitor 38 so as to display the retrieved records on the device image 164. To help the user trace the memory number, a footprint 128 may be highlighted on the kick count window 118.

Considering the enormous storage capacity of modem computers, it should be apparent to those of ordinary skill that the virtual device can store and display any suitable number of KickTimes records. However, for the purpose of illustration, the number of KickTime records is limited to ten in the present document.

The user may press the MODE button 114 to exit the kick memory mode and switch to a “play” mode. FIG. 3D is an exemplary device image 166 in a play mode for playing a heartbeat sound (or any other sound) recorded in the data storage device 26 as indicated in a text bubble 172. In this mode, the user may be able to play the fetal heartbeat sound, which is preferably a fetal heartbeat sound transferred from a Doppler instrument 44, by pressing down the EVENT button 112 for more than 0.15 seconds.

By default, the virtual device may include a prerecorded stock heart beat sound in the data storage 26. To overwrite the prerecorded stock heat beat sound, the Doppler instrument 44 may communicate a new heartbeat sound to the computer 10 via the communication interface 22. Alternatively, the heartbeat sound may be transferred to the computer by use of the CRSM 20 or through the network 46, such as the Internet. Further alternatively, the microphone 42 may be used to record a heartbeat sound. As depicted in FIG. 3D, a play icon 156 may be used to indicate the virtual device is currently operating in the play mode. The play icon 156 may flash during the playback of the sound recording or it may not be highlighted if the data storage device 26 does not have any recorded sound. The volume of the speaker 40 may be controlled by pressing down the EVENT button 112 for more than 2 seconds during which the volume may toggle between 25% and 100% of the maximum level. Then, the user may set the speaker volume to a desired level by releasing the EVENT button 112.

The user may press the MODE button 114 to exit the play mode and switch to a “record” mode. FIG. 3E shows an exemplary device image 168 in the record mode for recording the heartbeat sound to be played in the play mode. As illustrated, a record icon 158 may be displayed on the mode window 116 to indicate that the virtual device is currently operating in the record mode. Initially, the data storage device 26 may include a pre-recorded heartbeat sound. This may be erased when the user makes her first recording. Also, a new recording may automatically overwrite the previous one. The virtual device may record the heartbeat sound for 10 seconds. More specifically, the user may press down the EVENT button 112 for more than 0.5 seconds. Then, the input device driver 36 sends a signal to the kick counter 30 that subsequently sends a signal to the sound card driver 34 so that the sound card driver 34 records and stores the heartbeat sound in the data storage device 26. The record icon 158 may flash for 10 seconds from the start of recording and subsequently stay highlighted to indicate that the recording has completed. It should be apparent to those of skill in the art that the virtual device (or, equivalently, the kick counter 30) may be easily programmed to record more or less than 10 seconds. The counter may also be programmed to record more than one sound recording.

The user may press and hold the MODE button 114 in any mode to exit the current mode and switch to a “setup” mode. FIG. 3F shows an exemplary virtual image 170 in the setup mode for inputting information of the current stage of pregnancy into the virtual device. The setup mode has three submodes: “SetWeeks,” “SetDates,” and “SetLanguage” submodes. When the virtual device enters into the SetWeeks submode, the number representing the “Weeks Pregnant” in the week-and-day counter 130 may flash indicating that the virtual device is operating in the SetWeeks submode. The user may press the EVENT button 112 to set the weeks, where the weeks may loop from 0 to 42 and back to 0. The user may press the mode button to accept the weeks entered in the SetWeeks submode and enter the SetDays submode. In the SetDays submode, the number representing the “Days Pregnant” (in addition to the weeks pregnant) in the week-and-day counter 130 may flash and the user may press the EVENT button 112 to set the days, wherein the days may loop from 0 to 6.

The virtual device may be set to count forward from the inception of pregnancy as well as to count down until the completion of 40 weeks of pregnancy. If the user's input for the week-and-day counter 30 indicates that the current date is in the last 99 days of the pregnancy, the device image 170 may include an additional display of the count-down days on the day counter 132 as illustrated in FIG. 3F.

The user may press the mode button to accept the days entered and move into the SetLanguage submode. The words “DAYS TO GO” or “FALTAN_DIAS” of the day counter 132 may blink to indicate the currently selected language. The user may toggle between the languages by pressing the EVENT button 112 and select one by pressing the MODE button 114, this will also switch the virtual device back to the SetWeeks submode. Once a language is selected, the graphics on the windows 116, 118 and 120 may be displayed in the selected language.

When the user starts running the virtual device for the first time after installing the program, the virtual device may enter into the SetLanguage submode prompting the user to select a language. The user may exit the setup mode by pressing and holding the MODE button 114 in any submode. It should be apparent to those of ordinary skill that the virtual device may be designed to display other languages than English and Spanish. Also, the ordinary skill in the art would appreciate that the design engineer of the virtual device can easily change the shapes of the icons displayed on the display windows 116 and 118.

The data storage device 26 may also store other information including baby names, obstetrical data such as fetal development milestones, personal medical and emergency information, medical visits, obstetrical provider's contact details, gestational age specific recommendation, organizer, calendar, chronometer and pediatric data, such as pediatric growth chart. It should be apparent to those of ordinary skill that the stored data may be manipulated and accessed by various user programs including the kick counter 30. For example, the Microsoft Excel™ program, may access the data stored in the data storage device 26 and plot/print the KickTime as a function of time. The user may be able to select an option to view the kick times in a graphical format. There may also be the option to print this data or store it for future reference. The option may be provided to e-mail the kick count sessions, or a graph of kick count sessions to the doctor for review. An option could be provided to e-mail sound recordings to family and friends. These options could be offered if the user clicks the right mouse button to obtain a drop down menu of options. As the conventional method for providing a drop down menu upon click of the right mouse button is well known in the art, detailed description is not given for simplicity. Also, the data may be transferred to or from another computer through the communication interface 22 or CRSM 20, and printed by the printer 15.

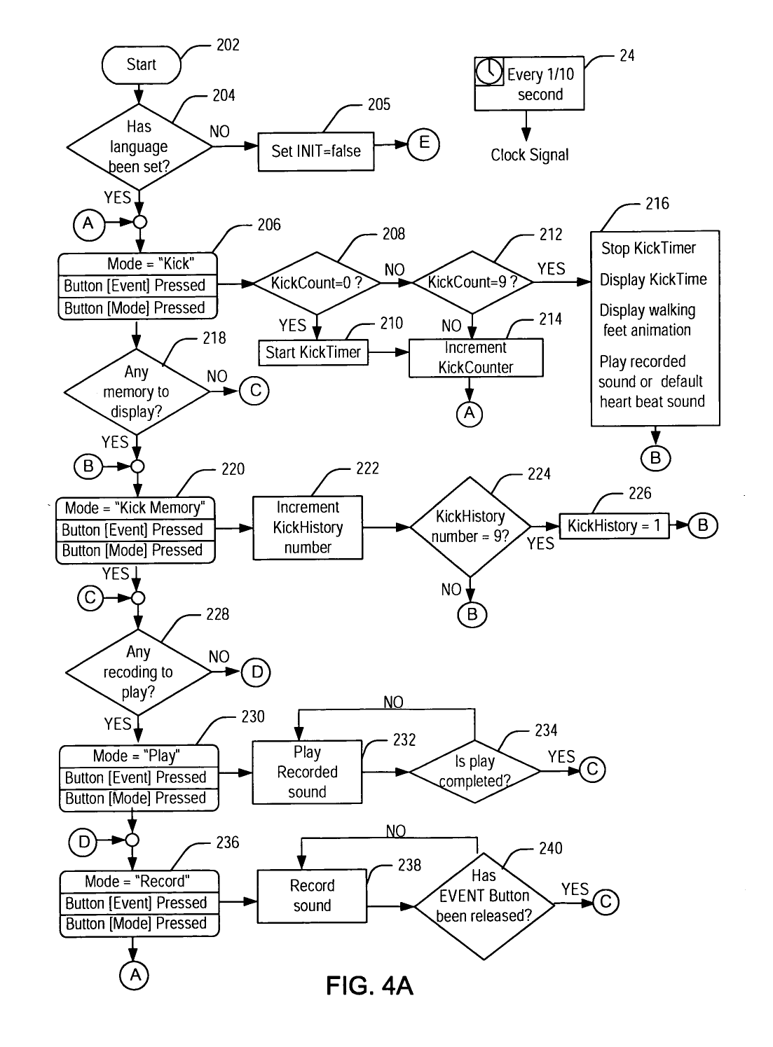

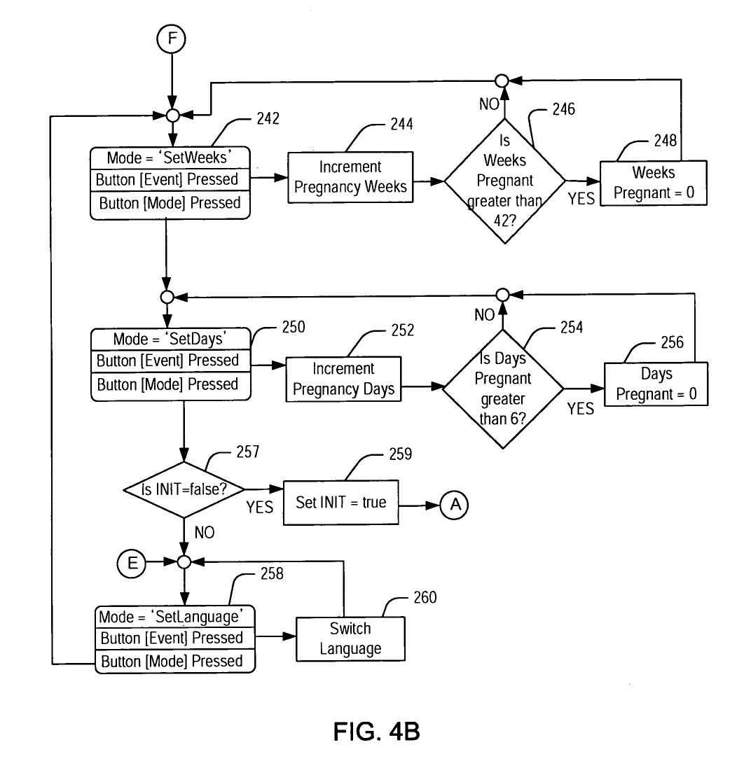

FIGS. 4A-4B show a flow chart illustrating the operational sequence of the modes depicted in connection with FIGS. 3A-3F. The process may start in a state 202. Next, in a state 204, the kick counter 30 may check if the user has selected a language. Upon negative answer to the state 204, the INIT (short for initialization) may be set to false in a state 205, i.e. the device has not been initialized and will automatically default to the SetLanguage submode. Subsequently, the process may advance to a state 258 (shown and illustrated in connection with FIG. 4B). Otherwise, the user may press the MODE button 114 to proceed to a state 206. It is noted that the timer 24 may send a clock signal every 1/10 second.

The state 206 may correspond to the kick mode. If the user presses the EVENT button 112 to record a kick, the kick counter 30 may determine if the kick is the first one of the current session. Upon positive answer to the state 208, the kick counter 30 may start the timer 24 in a state 210, where the timer 24 may operate to measure the KickTime of the current session. If the answer to the state 208 is negative, the process may advance to a state 212.

In the state 212, the kick counter 30 may determine if the KickCount is equal to 9 and the current kick is therefore the 10^{th} kick of the current session. Upon positive answer to the state 212, the kick counter 30 may stop the timer 24 and play the animation as described in connection with FIG. 3B. The animation may include displaying the KickTime on the time display 124, displaying a walking footprint animation 128 and playing a recorded heartbeat sound 152. Then, the process may proceed to a state 220. Upon negative answer to the state 212, the kick counter 30 may increment the kick count by one. Subsequently, the process may proceed to the state 206 to cause the virtual device to stay in the kick mode.

If the user presses the MODE button 114 in the state 206, the process may advance to a state 218. In the state 218, the kick counter 30 may determine whether the user has logged a KickTime record to display, i.e., the user has completed one ten kick session. If a ten KickTime record is stored in the data storage device 26 (FIG. 1), the process may advance to a state 220. Otherwise the process may proceed to a state 228.

The state 220 may correspond to the kick memory (or, shortly, memory) mode. As described with reference to FIG. 3C, the user may press the EVENT button 112 to display the ten KickTime records sequentially, one at a time. The EVENT button 112 may be used to scroll through the ten records. For each record displayed, a corresponding footprint 128 may be displayed on the kick count window 118. The record number for the corresponding KickTime record may be displayed on the record number display 126. Also, the time display 124 may be used to indicate the KickTime. Each time the user presses the EVENT button 112, the kickhistory number (or equivalently, record number) may be increased by one in a state 222. Then the process may proceed to a state 224.

In the state 224, the kick counter 30 may determine if the current kickhistory number equals nine, i.e., the currently displayed KickTime record is the tenth one. Upon negative answer to the state 224, the process may proceed to the state 220. Otherwise, the kick counter 30 may reset the kickhistory number to 1. Subsequently, the process may advance to the state 220.

In the state 220, the user may press the MODE button 114 causing the process to advance to a state 228. In the state 228, the decision is made as to whether the data storage device 26 has any sound recording to be played. Upon negative answer to the state 228, the process may proceed to a state 236. Otherwise, the process may proceed to a state 230.

The state 230 may correspond to the play mode. In the state 230, the user may press the EVENT button 112, causing the process to proceed to a state 232. In the state 232, the kick counter 30 may play the recorded fetal heartbeat sound. Next, in a state 234, the kick counter 30 may determine whether the play has completed. Upon positive answer to the state 234, the process may advance to the state 228. If the answer to the state 234 is NO, the process may proceed to the state 232.

The state 236 may correspond to the record mode. The kick counter 30 may reach state 236 by pressing the MODE button in state 230, or by answering negatively to state 228. In the state 236, the user may press the EVENT button 112, causing the process to proceed to a state 238. In the state 238, the kick counter 30 may record the fetal heartbeat sound from the Doppler instrument 44 by pressing and holding the EVENT button 112. More specifically, the kick counter 30 may operate the sound card driver 34 to receive the fetal heartbeat sound and to store the received sound into the data storage device 26. Next, in a state 240, the kick counter 30 may determine whether the EVENT button 112 has been released. Upon positive answer to the state 240, the process may advance to the state 228. If the answer to the state 240 is NO, the process may proceed to the state 238.

The user may press the MODE button 114 in the state 236, causing the process to proceed back to state 206 in FIG. 4A. The user may press and hold the MODE button in any state to cause the process to proceed to a state 242 in FIG. 4B. The state 242 may correspond to the SetWeeks submode. In the state 242, the user may press the EVENT button 112 causing the process to proceed to a state 244. In the state 244, the kick counter 30 may increase the Weeks Pregnant in the week-and-day counter 130 by one each time the user presses the EVENT button 112. Next, in a state 246, a determination may be made as to whether the Weeks Pregnant is greater than forty two. Upon negative answer to the state 246, the process may proceed to the state 242. Otherwise, the process may advance to a state 248. In the state 248, the Weeks Pregnant may be set to zero. Then, the process may proceed to the state 242. The user may press the MODE button 112 in the state 242, causing the process to proceed to the state 250.

The state 250 may correspond to the SetDays submode. In the state 250, the user may press the EVENT button 112 causing the process to proceed to a state 252. In the state 252, the kick counter 30 may increase the Days Pregnant in the week-and-day counter 130 by one each time the user presses the EVENT button 112. Next, in a state 254, a determination may be made as to whether the DueDays is greater than six. Upon negative answer to the state 254, the process may proceed to the state 250. Otherwise, the process may advance to a state 256. In the state 256, the Days Pregnant may be set to zero. Then, the process may proceed to the state 250. The user may press the MODE button 114 in the state 250, causing the process to proceed to the state 257. In the state 257, a determination may be made as to whether the INIT is set to false, i.e. whether the initialization has been carried out. Upon positive answer to the state 257, the process may proceed to a state 259. In the state 259, the INIT is set to true. Then, the process may proceed to the state 206. If the answer to the state 257 is negative, the process may also proceed to a state 258.

The state 258 may correspond to the SetLanguage submode. In the state 258, the user may press the EVENT button 112 causing the process to proceed to a state 260. In the state 260, the user may select a language as described in connection with FIG. 3F. Then, the process may advance to the state 258. The user may press the MODE button 114 in the state 258, causing the process to proceed to back to the state 242. The user may press and hold the MODE button during any submode to exit the setup and return to state 206.

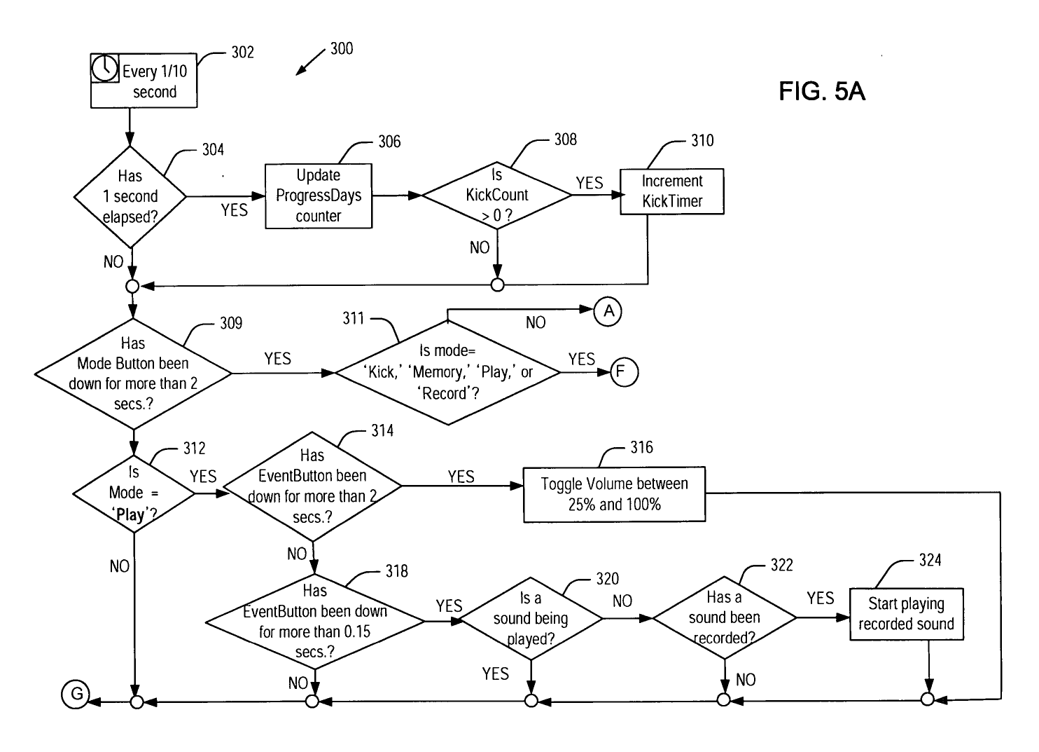

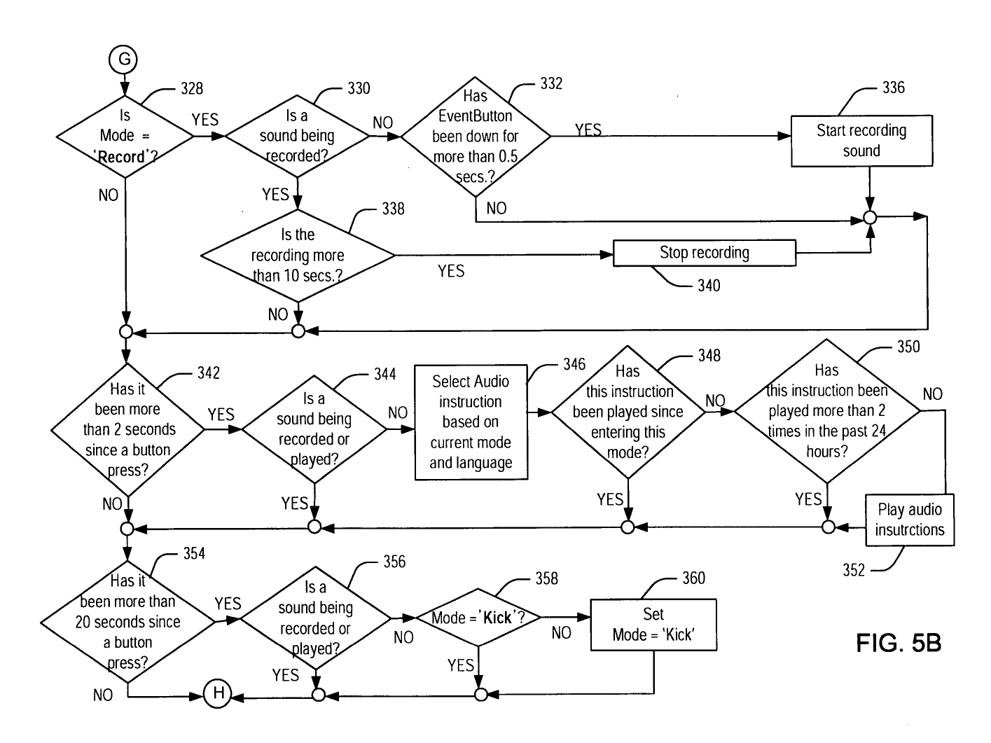

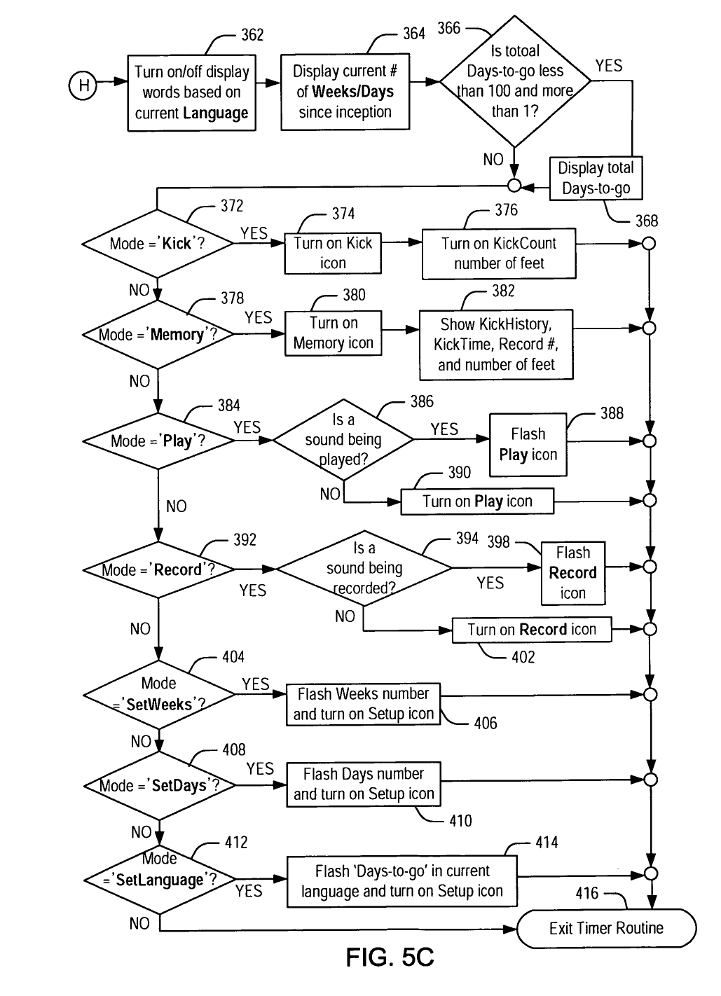

FIGS. 5A-5C show a system flowchart 300 of the virtual device depicted in connection with FIGS. 1-3F. The process may start at every 1/10 second in a state 302, where the timer 24 (FIG. 1) may provide the clock time. Next, the process may advance to a state 304, where a determination may be made as to whether one second has elapsed. Upon positive answer to the state 304, the progress may advance to a state 306. In the state 306, the kick counter 30 may update the ProgressDays counter, where the ProgressDays collectively refer to the “Weeks and Days Pregnant” of the week-and-day counter 130 and the count-down of days-to-go of the day counter 132. Next, in a state 308, another determination is made as to whether the KickCount is greater than zero, i.e., the user has started recording the kicks in a session. Upon positive answer to the state 308, the KickTimer may be incremented in a state 310. Then, the process may advance to a state 309. If the answer to the state 308 is negative or the answer to the state 304 is negative, the process may proceed to the state 309.

In the state 309, a decision is made as to whether the MODE button has been held down for more than 2 seconds. Upon positive answer to state 309, the process may advance to state 311, where a decision is made as to whether the mode is the ‘kick’, ‘memory’, ‘play’, or ‘record’ mode. Upon negative answer to the state 311, the process may proceed to state 206. Upon positive answer to the state 311, the process may proceed to state 242 in FIG. 4B. In state 309, if the answer is negative, the process proceeds to state 312.

In the state 312, a decision may be made as to whether the current mode is the play mode. Upon positive answer to the state 312, the process may proceed to a state 314.

In the state 314, the kick counter 30 may determine if the user has pressed down the EVENT button 112 for more than 2 seconds. If the answer to the state 314 is YES, the process may proceed to a state 316. In state 316, the speaker volume may toggle between 25% and 100% of the maximum level.

If the answer to state 314 is negative, the process may proceed to state 318. In the state 318, the input device driver 36 may determine if the user has pressed down the EVENT button 112 for more than 0.15 seconds. If the answer to the state 318 is YES, the process may proceed to a state 320. In the state 320, a determination may be made as to whether the data storage device 26 is currently playing a heartbeat sound. Upon negative answer the process may proceed to state 322. In state 322 the process may determine if there is a sound recording to play. Upon positive answer, the process may proceed to state 324. The kick counter 30 may play the sound through the speaker 40 in a state 324. Then, the process may proceed to a state 328 (FIG. 5B). If the answer to the state 320 is positive or the answer to the state 322 is negative, the process may advance to the state 328. Upon negative answer to the state 312, the process may proceed to a state 328.

In the state 328, a decision may be made as to whether the current mode is the record mode. Upon positive answer to the state 328, the process may proceed to a state 330. In the state 330, a determination may be made as to whether a sound is being recorded. If the answer to the state 330 is NO, the process may proceed to a state 332. In the state 332, the kick counter 30 may determine if the user has pressed down the EVENT button 12 for more than 0.5 seconds. If the user has pressed down the EVENT button 112 more than 0.5 seconds, the process may proceed to a state 336 to start recording a sound. Then, the process may proceed to a state 342. If the answer to the state 332 is negative, the process may also proceed to the state 342.

Upon positive answer to the state 330, the process may proceed to a state 338. In the state 338, a determination may be made as to whether the recording is more that 10 seconds. If the answer to the state 338 is YES, the process may proceed to a state 340 to stop recording. Subsequently, the process may proceed to the state 342. If the answer to the state 328 is negative, the process may also proceed to the state 342.

In the state 342, the kick counter 30 may determine if more than 2 seconds has elapsed since a button has been pressed. If the answer to the state 342 is YES, the process may proceed to a state 344. In the state 344, a determination may be made as to whether a sound is being recorded or played. If the answer to the state 344 is NO, the process may proceed to a state 346. In the state 346, the kick counter 30 may select the audio instruction sound based on the current mode and language. Then, the process may proceed to a state 348. In state 348, a determination may be made as to whether the selected instructions have already been played since entering the current mode. Upon negative answer to the state 348, the process may proceed to state 350. In state 350, a determination may be made as to whether the instructions have been played more than 2 times in the past 24 hours. If the answer to state 350 is negative, the process may proceed to state 352 and play the selected audio instructions. If the answer to the state 342 is NO or the answers to the states 348, 350 are YES, the process may also proceed to the state 354.

In the state 354, the kick counter 30 may determine if more than 20 seconds has elapsed since a button has been pressed. If the answer to the state 354 is YES, the process may proceed to a state 356. In the state 356, a determination is made whether a recording is being played. If the answer to the state 356 is NO, the process may proceed to a state 358. In the state 358, a determination is made whether the current mode is the kick mode. If the answer to the state 350 is NO, the process may proceed to a state 360. In the state 360, the current mode is set to the kick mode. Then, the process may advance to a state 362 (FIG. 5C). If the answer to the state 354 is NO or the answer to the state 356 is YES or the answer to the state 358 is YES, the process may proceed to a state 362.

In the state 362, the kick counter 30 may turn on/off the displayed words based on the current language. Next, in a state 364, numbers of weeks and days may be displayed on the week-and-day counter 130. Then, the process may advance to a state 366. In the state 366, a determination is made whether the countdown days-to-go is less than 100 and more than 1. If the answer to the state 366 is YES, the process may proceed to the state 368 to display the countdown days-to-go on the day counter 132. Then, the process may proceed to a state 372. If the answer to the state 366 is NO, the process may also proceed to the state 372.

In the state 372, a determination is made as to whether the current mode is the kick mode. If the answer to the state 372 is YES, the kick icon 150 (FIG. 3A) may be turned on (or equivalently, highlighted) in a state 374. Subsequently, in a state 376, footprints 128 may be displayed on the kick count window 118. Then, the process may proceed to a state 416. If the answer to the state 372 is NO, the process may proceed to a state 378.

In the state 378, a determination is made as to whether the current mode is the kick memory (or, shortly, memory) mode. If the answer to the state 378 is YES, the memory icon 154 (FIG. 3C) may be turned on in a state 380. Subsequently, in a state 382, the kickhistory for each KickTime record may be displayed, where the kickhistory may include a KickTime, a record number and a footprint corresponding to the record number. Then, the process may proceed to a state 416. If the answer to the state 378 is NO, the process may proceed to a state 384.

In the state 384, a determination is made as to whether the current mode is the play mode. If the answer to the state 384 is YES, a determination may be made as to whether a sound is being played in a state 386. If the answer to the state 386 is YES, the play icon 156 (FIG. 3D) may flash in a state 388. Subsequently, the process may proceed to the state 416. If the answer to the state 386 is NO, the play icon 156 may be highlighted in a state 390. Then, the process may proceed to the state 416. If the answer to the state 384 is NO, the process may proceed to a state 392.

In the state 392, a determination is made as to whether the current mode is the record mode. If the answer to the state 392 is YES, a determination may be made as to whether a sound is being recorded in a state 394. If the answer to the state 394 is YES, the process may proceed to a state 398. In the state 398, the record icon 158 may flash. Then, the process may proceed to the state 416. If the answer to the state 394 is NO, the record icon 158 may be highlighted in a state 402. Subsequently, the process may proceed to the state 416. If the answer to the state 392 is NO, the process may proceed to a state 404.

In the state 404, a determination is made as to whether the current mode is the SetWeeks submode. If the answer to the state 404 is YES, the Weeks Pregnant of the week-and-day counter 130 may flash in a state 406 and the ‘setup’ icon may be highlighted. Then, the process may proceed to the state 416. If the answer to the state 404 is NO, the process may proceed to a state 408.

In the state 408, a determination is made whether the current mode is the SetDays submode. If the answer to the state 408 is YES, the DueDays of the week-and-day counter 130 may flash in a state 410 and the ‘setup’ icon may be highlighted. Then, the process may proceed to the state 416. If the answer to the state 408 is NO, the process may proceed to a state 412.

In the state 412, a determination is made whether the current mode is the SetLanguage submode. If the answer to the state 414 is YES, the word “days to go” of the day counter 130 may flash in the current language in a state 414 and the ‘setup’ icon may be highlighted. Then, the process may proceed to the state 416. If the answer to the state 412 is NO, the process may proceed to the state 416. In the state 416, the process for every 0.1 second started in the state 302 in FIG. 5A may exit.

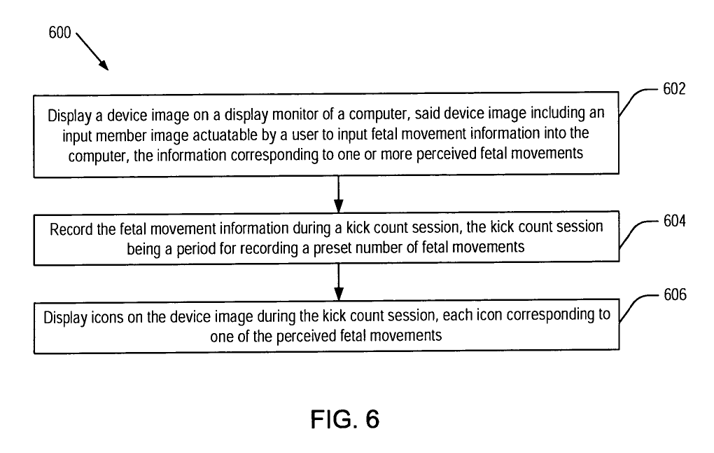

FIG. 6 is a flow chart shown at 600 illustrating the steps that may be carried out to count kicks using the virtual device or kick counter program according to the present invention. In a state 602, the computer 10 may display a device image on a display monitor, where the device image includes the EVENT button 112 (or, equivalently, an image of an input member) actuatable by the user to input fetal movement information into the computer 10. The fetal movement information may correspond to one or more perceived fetal movements. Then, the virtual device may record the fetal movements up to a preset number, preferably ten, in the kick count session in a state 602. During the kick count session, the virtual device may display icons, where one additional icon is displayed each time the user presses the EVENT button 112 in a state 606. As a consequence, the user can count the perceived fetal movements recorded in the kick count session by counting the displayed icons.

It should be understood, of course, that the foregoing relates to exemplary embodiments of the invention and that modifications may be made without departing from the spirit and scope of the invention as set forth in the following claims.

What is claimed is:

1. A method to be performed by a computer for charting fetal movements, comprising:
displaying a device image on a display monitor of the computer, said device image including an input member image actuatable by a user to input fetal movement information into the computer, the information corresponding to one or more perceived fetal movements;
recording the fetal movement information during a kick count session, the kick count session being a period for recording a preset number of fetal movements; and
displaying icons on the device image during the kick count session, each icon corresponding to one of the perceived fetal movements,
whereby the user is able to count the perceived fetal movements recorded during the kick count session by counting the icons displayed on the device image during the kick count session.

2. A method to be performed by a computer for charting fetal movements as recited in claim 1, further comprising:
measuring the duration of the period, KickTime; and
storing the KickTime in a data storage.

3. A method to be performed by a computer for charting fetal movements as recited in claim 2, further comprising:
displaying, on the device image, the KickTime stored in said data storage when the user actuates the input member image.

4. A method to be performed by a computer for charting fetal movements as recited in claim 2, wherein said computer is coupled to a Doppler instrument, said method further comprising:
receiving a fetal heartbeat sound from said Doppler instrument; and
storing the heartbeat sound into said data storage.

5. A method to be performed by a computer for charting fetal movements as recited in claim 4, wherein each of the icons is in the form of a footprint, the method further comprising:
upon completion of the kick count session, sequentially displaying the icons on the device image to perform an animation of feet walking; and
playing the heartbeat sound by use of a speaker means during the animation.

6. A method to be performed by a computer for charting fetal movements as recited in claim 2, wherein said computer is coupled to an audio member, said method further comprising:
receiving a sound using the audio member; and
storing the sound in said data storage.

7. A method to be performed by a computer for charting fetal movements as recited in claim 6, wherein the sound is one selected from the group consisting of heartbeat sound from a Doppler instrument, baby's first cry and baby's first words.

8. A method to be performed by a computer for charting fetal movements as recited in claim 6, the method further comprising:
receiving a user's voice command by use of the audio member; and
recognizing the received user's voice command.

9. A method to be performed by a computer for charting fetal movements as recited in claim 1, wherein the information further includes pregnancy progress data in weeks and days, the method further comprising:
calculating a countdown of days-to-go using the pregnancy progress data; and
displaying the countdown on the device image if the countdown is less than a predetermined number.

10. A method to be performed by a computer for charting fetal movements as recited in claim 1, further comprising:
causing said device image to have multiple language settings.

11. A method to be performed by a computer for charting fetal movements as recited in claim 1, further comprising:
displaying a digital image of a baby's ultrasound scan on the device image.

12. A method to be performed by a computer for charting fetal movements as recited in claim 1, wherein the computer includes a communication interface for communicating with an electronic device.

13. A method to be performed by a computer for charting fetal movements as recited in claim 12, wherein the electronic device is one selected from the group consisting of a computer and a personal digital assistant (PDA).

14. A method to be performed by a computer for charting fetal movements as recited in claim 12, wherein the communication interface is operable to communicate with a wireless device.

15. A method to be performed by a computer for charting fetal movements as recited in claim 2, further comprising:
storing one or more audio instructions for use of the device image in said data storage; and
providing the stored audio instructions upon request of the user.

16. A method to be performed by a computer for charting fetal movements as recited in claim 1, wherein said computer includes an ultrasound device for sensing and imaging the perceived fetal movements.

17. A computer readable medium carrying one or more sequences of pattern data for charting fetal movements, wherein execution of the one or more sequences of pattern data by one or more processors causes the one or more processors to perform the steps of:
displaying a device image on a display monitor of a computer, said device image including an input member image actuatable by a user to input fetal movement information into the computer, the information corresponding to one or more perceived fetal movements;
recording the fetal movement information during a kick count session, the kick count session being a period for recording a preset number of fetal movements; and
displaying icons on the device image during the kick count session, each icon corresponding to one of the perceived fetal movements,
whereby the user is able to count the perceived fetal movements recorded during the kick count session by counting the icons displayed on the device image during the kick count session.

18. A computer readable medium as recited in claim 17, wherein execution of the one or more sequences of pattern data by one or more processors causes the one or more processors to perform the additional steps of:
measuring the duration of the period, KickTime; and
storing the KickTime in a data storage.

19. A computer readable medium as recited in claim 18, wherein execution of the one or more sequences of pattern data by one or more processors causes the one or more processors to perform the additional step of:
displaying, on the device image, the KickTime stored in said data storage when the user actuates the input member image.

20. A computer readable medium as recited in claim 18, wherein the computer is coupled to a Doppler instrument, and wherein execution of the one or more sequences of pattern data by one or more processors causes the one or more processors to perform the additional steps of:
receiving a fetal heartbeat sound from the Doppler instrument; and
storing the heartbeat sound into said data storage.

21. A computer readable medium as recited in claim 20, wherein each of the icons is in the form of a footprint, and wherein execution of the one or more sequences of pattern data by one or more processors causes the one or more processors to perform the additional steps of:
upon completion of the kick count session, sequentially displaying the icons on the device image to perform an animation of feet walking; and
playing the heartbeat sound by use of a speaker means during the animation.

22. A computer readable medium as recited in claim 18, wherein said computer is coupled to an audio member, and wherein execution of the one or more sequences of pattern data by one or more processors causes the one or more processors to perform the additional steps of:
receiving a sound using the audio member; and
storing the sound in said data storage.

23. A computer readable medium as recited in claim 22, wherein execution of the one or more sequences of pattern data by one or more processors causes the one or more processors to perform the additional steps of:
receiving a user's voice command by use of the audio member; and
recognizing the received user's voice command.

24. A computer readable medium as recited in claim 17, wherein the information further includes pregnancy progress data in weeks and days, and wherein execution of the one or more sequences of pattern data by one or more processors causes the one or more processors to perform the additional steps of:
calculating a countdown of days-to-go using the pregnancy progress data; and
displaying the countdown on the device image if the countdown is less than a predetermined number.

25. A computer readable medium as recited in claim 17, wherein execution of the one or more sequences of pattern data by one or more processors causes the one or more processors to perform the additional step of:
causing the device image to have multiple language settings.

26. A computer readable medium as recited in claim 17, wherein execution of the one or more sequences of pattern data by one or more processors causes the one or more processors to perform the additional step of:
displaying an image of a baby's ultrasound scan on the device image.

27. A computer readable medium as recited in claim 18, wherein execution of the one or more sequences of pattern data by one or more processors causes the one or more processors to perform the additional steps of:
storing one or more audio instructions for use of the device image in the data storage; and
providing the stored audio instructions upon request of the user.

28. A computer readable medium as recited in claim 17, wherein the computer is coupled to an ultrasound device, and wherein execution of the one or more sequences of pattern data by one or more processors causes the one or more processors to perform the additional step of:
sensing and imaging the perceived fetal movements by use of the ultrasound device.
